(19) United States
(12) Patent Application Publication
(10) Pub. No.: US 2024/0131098 A1
(43) Pub. Date: Apr. 25, 2024

(54) PHARMACEUTICAL COMPOSITION AND HEALTH FUNCTIONAL FOOD CONTAINING EXTRACT OF FRUITING BODY OF GANODERMA LUCIDUM AS ACTIVE INGREDIENT FOR PREVENTION OR TREATMENT OF THROMBOSIS

(71) Applicant: Andong National University Industry-Academic Cooperation Foundation, Andong-si (KR)

(72) Inventor: Ho Yong SOHN, Daegu (KR)

(73) Assignee: Andong National University Industry-Academic Cooperation Foundation, Andong-si, Gyeongsangbuk-do (KR)

(21) Appl. No.: 18/173,316

(22) Filed: Feb. 23, 2023

(30) Foreign Application Priority Data
Oct. 6, 2022 (KR) 10-2022-0127571

(51) Int. Cl. A61K 36/074; A23L 31/00; A61P 7/02; C12N 1/14

(52) U.S. Cl. CPC A61K 36/074; A23L 31/00; A61P 7/02; C12N 1/14

(57) ABSTRACT

Proposed are pharmaceutical compositions and health functional foods containing a Ganoderma lucidum fruiting body extract as an active ingredient for preventing, treating, or ameliorating thrombosis. The extract exhibits strong antithrombotic activity by inhibiting thrombosis-related enzymes and blood coagulation factors, has excellent thermal stability, and exhibits no loss of blood coagulation factor inhibitory effect and thrombogenic enzyme inhibitory effect even in acidic conditions of pH 2 and in plasma. Therefore, the Ganoderma lucidum fruiting body extract is expected to be used for the prevention and treatment of thrombosis, which causes ischemic stroke and hemorrhagic stroke, by improving blood circulation. The Ganoderma lucidum fruiting body extract is very useful in the pharmaceutical and food industries.

### CROSS REFERENCE TO RELATED APPLICATION

The present application claims priority to Korean Patent Application No. 10-2022-0127571, filed Oct. 6, 2022, the entire contents of which is incorporated herein for all purposes by this reference.

### BACKGROUND OF THE DISCLOSURE

### Field of the Disclosure

The present disclosure relates to pharmaceutical compositions and health functional foods containing an extract of a fruiting body of Ganoderma lucidum as an active ingredient for preventing, treating, or ameliorating thrombosis. More particularly, the present disclosure relates to pharmaceutical compositions and health functional foods containing a Ganoderma lucidum fruiting body ethanol extract as an active ingredient for preventing, treating, or ameliorating thrombosis through blood clotting inhibition.

### Description of the Related Art

Blood, as a component of the human body, has various important functions: transporting oxygen, nutrients, and waste; buffering; regulating body temperature, osmotic pressure, ion balance, water constant, humoral control, blood pressure; and protecting the body. In normal blood circulation, the blood coagulation reaction system and the blood clot dissolution reaction system are complementarily regulated to facilitate blood circulation. Among them, it has been reported that the blood coagulation reaction system acts in a manner that a blood coagulation system is activated when platelets adhere to the blood vessel wall and aggregate each other to form a platelet thrombus, and thus a fibrin thrombus is formed around the platelet aggregation.

In fibrin thrombus formation, thrombin, which is involved in fibrin coagulation through multi-step reactions of numerous blood coagulation factors, is activated to convert fibrinogen to fibrin monomers. The fibrin monomers are polymerized by calcium, bind to platelets and endothelial cells, and form fibrin polymers cross-linked by factor XIII permanent blood clots while forming fibrin polymers cross-linked by factor XIII, resulting in permanent blood clots. In addition, thrombin plays a pivotal role in the formation of blood clots, such as promoting blood coagulation by activating platelets, factor V, and factor VII. Therefore, substances that inhibit the activity of thrombin can be used as very useful preventive and therapeutic agents for various thrombotic diseases caused by excessive blood coagulation.

In the endogenous thrombogenesis pathway, sequential activation of factor XII, factor XI, factor IX, and factor X followed by activation of prothrombin is known to finally activate thrombin. Therefore, specific inhibition of blood coagulation factors is also an important development target for thrombotic disease treatments. Until now, various anticoagulants such as heparin, coumarin, aspirin, and urokinase, anticoagulants, antiplatelet agents, and thrombolytic agents have been used for the prevention and treatment of thrombotic diseases. However, since these agents are very expensive and causes problems such as hemorrhagic side effects, gastrointestinal disorders, and hypersensitivity reactions, their use is limited.

On the other hand, Ganoderma lucidum is an annual plant that sprouts from broad-leaved trees in summer and is also called Yeongjicho ( [symbol] ), Jicho ( [symbol] ), eternal herb ( [symbol] ), etc. The stem is about 10 cm high, the hat is heart-shaped or circular, and the surface of the hat and handle has a gloss like lacquer. When young, it turns into egg yolk white, yellowish brown, or reddish brown, and when it is aged, the body is like leathery cork, which is hard and glossy reddish brown or purple brown. The mushroom body is composed of two layers in which the upper layer is almost white, and the lower layer of the tube part is light yellow. The hat is semi-circular, kidney-shaped, or fan-shaped, with a flat surface and concentric ring holes.

Because Ganoderma lucidum is hard like a tree, it is impossible to directly eat it as it is. It is ground into powder or dried for medicinal purposes. It is common to consume Ganoderma lucidum as tea made by brewing the powder or dried fragments of the Ganoderma lucidum. In oriental medicine, Ganoderma lucidum is used for treating nervous breakdown, heart diseases, high blood pressure, and various cancers because it has effects such as health enhancing effect, antitussive effect, and detumescence inducing effect.

There are known studies regarding Ganoderma lucidum: “Cancer Cell Growth Inhibitory Effect of Lanostane-type Triterpenoid Isolated from Ganoderma Lucidum (Kim Dong-Hwa et al., 2020, Journal of Herbal Medicine 51: 36-40), “Immunity Enhancing Activity of Lanostane-Type Triterpenoids Isolated from Fruiting Bodies Of Ganoderma Lucidum” (Pu, D. B et al., 2017. Highly oxygenated lanostane-type triterpenoids and their bioactivity from the fruiting body of Ganoderma lucidum. Fitoterapia 119: 1-7), and “Antifungal Active Substance” (Pu, D. B et al, 2019. Triterpenoids from Ganoderma lucidum: A Class of Sensitizers of FLC-Resistant Candida albicans to Fluconazole. J. Natural products 82: 2067-2077). Recently, “Artificial Culture and Nutritional Evaluation of Wild Ganoderma Lucidum” (Yu, Changxia et al, 2020, J. Science Society of Thailand 46: 548) has been reported.

There are patent documents related to Ganoderma lucidum: Korean Patent No. 10-0963826 [New Ganoderma lucidum and Cultivation Method Thereof]; Korean Patent No. 10-0931526 [Fermented Food Using Ganoderma lucidum Mycelium and Manufacturing Method Thereof]; Korean Patent No. 10-1002256 [Abalone Drying Method Using Ganoderma lucidum and Far-Infrared Rays]; Korean Patent No. 10-1303544 [Natural Dye for Clothing and Manufacturing Method Thereof]; Korean Patent No. 10-1477078 [Natural Deodorant and Disinfectant Containing Herbal Ingredients and Manufacturing Method Thereof]; Korean Patent No. 10-1541872 [Livestock Feed Using Food Waste Leachate and Manufacturing Method Thereof]; Korean Patent No. 10-1625937 [Seed Treatment Method and Seeds Treated Thereby]; and Korean Patent No. 10-1996384 [Method of Manufacturing Functional Cheonggukjang Block].

However, until now, no research or patent literatures related to the strong anticoagulant activity of Ganoderma lucidum fruiting body extract has been known.

### LITERATURE OF RELATED ART

### Patent Literature

(Patent Literature 0001) Korean Patent No. 10-1875657

### Non-Patent Literature

(Non-patent Literature 0001) Dong-Hwa KIM et al., 2020, Biopharmaceutical Journal 51: 36-40, Cancer Cell Growth Inhibitory Effect of Lanostane-type Triterpenoid isolated from Ganoderma lucidum.

### SUMMARY OF THE DISCLOSURE

The present disclosure has been made to solve the problems occurring in the related art, and the present disclosure provides a pharmaceutical composition and health functional food containing Ganoderma lucidum fruiting body extract as an active ingredient for preventing, treating, or ameliorating thrombosis by intensive blood clotting inhibitory effect.

In order to solve the above problems, the present disclosure provides a pharmaceutical composition containing Ganoderma lucidum fruiting body extract as an active ingredient for preventing or treating thrombosis.

It is preferable that the Ganoderma lucidum fruiting body extract is an ethanol extract of a Ganoderma lucidum fruiting body.

In addition, the present invention provides a blood coagulation inhibitor including a Ganoderma lucidum fruiting body extract as an active ingredient.

It is preferable that the Ganoderma lucidum fruiting body extract is an ethanol extract of a Ganoderma lucidum fruiting body.

In addition, the present disclosure provides a health functional food including Ganoderma lucidum fruiting body extract for prevention or ameliorating thrombosis.

It is preferable that the Ganoderma lucidum fruiting body extract is an ethanol extract of a Ganoderma lucidum fruiting body.

The Ganoderma lucidum fruiting body extract as an active ingredient of the pharmaceutical composition and health functional food for preventing or treating thrombosis, of the present disclosure, exhibits strong antithrombotic activity by inhibiting thrombosis-related enzymes and blood coagulation factors, has excellent thermal stability, and exhibits no loss of blood coagulation factor inhibitory effect and thrombogenic enzyme inhibitory effect even in acidic conditions of pH 2 and in plasma. Therefore, the Ganoderma lucidum fruiting body extract is expected to be used for the prevention and treatment of thrombosis, which causes ischemic stroke and hemorrhagic stroke, by improving blood circulation. In addition, the Ganoderma lucidum fruiting body extract does not exhibit toxicity when used for food. In addition, the Ganoderma lucidum fruiting body extract can be processed into various forms such as extracts, powders, pills, tablets, etc. That is, the Ganoderma lucidum fruiting body extract can be prepared in a form that can be conveniently taken at all times. Therefore, the Ganoderma lucidum fruiting body extract is very useful in the pharmaceutical and food industries.

### BRIEF DESCRIPTION OF THE DRAWINGS

FIG. 1 is a photographic view of Ganoderma lucidum fruiting body of the present disclosure and powder thereof.

### DESCRIPTION OF PREFERRED EMBODIMENTS

Hereinafter, the present disclosure will be described in detail.

In order to verify the antithrombotic efficacy of Ganoderma lucidum, the inventors of the present disclosure prepared an ethanol extract of the fruiting body of Ganoderma lucidum in a predetermined way, and then assayed the antithrombotic activity. As a result, the inventors confirmed that the extract had excellent antithrombotic activity, thermal stability, and acid stability and have tried to use the Ganoderma lucidum fruiting body extract as a pharmaceutical composition and health functional food for preventing, treating, or ameliorating thrombosis.

Specifically, Specifically, in order to develop pharmaceutical compositions and health functional foods for preventing, treating, or ameliorating thrombosis using Ganoderma lucidum, which is known to be effective in various diseases such as skin diseases, bacterial infectious diseases, diseases related to vascular and circulatory systems, diseases related to digestive and metabolic systems, etc., the inventors prepared various solvent extracts from the fruiting body of Ganoderma lucidum, and evaluated the direct thrombin inhibition (thrombin time), prothrombin inhibition (prothrombin time), and active partial thromboplastin time (aPTT) of the extracts. As a result, it was confirmed that the Ganoderma lucidum fruiting body extract had thrombogenesis inhibitory activity attributed to excellent thrombin inhibition, prothrombin inhibition, and blood coagulation factor inhibition.

Therefore, the present disclosure provides a pharmaceutical composition containing Ganoderma lucidum fruiting body extract as an active ingredient for preventing or treating thrombosis.

It is preferable that the Ganoderma lucidum fruiting body extract is an ethanol extract of a Ganoderma lucidum fruiting body.

In addition, the present invention provides a blood coagulation inhibitor including a Ganoderma lucidum fruiting body extract as an active ingredient.

It is preferable that the Ganoderma lucidum fruiting body extract is an ethanol extract of a Ganoderma lucidum fruiting body.

In addition, the present disclosure provides a health functional food including Ganoderma lucidum fruiting body extract for prevention or ameliorating thrombosis.

It is preferable that the Ganoderma lucidum fruiting body extract is an ethanol extract of a Ganoderma lucidum fruiting body.

Hereinafter, a method of preparing and testing the efficacy of the Ganoderma lucidum fruiting body extract of the present disclosure will be described in more detail.

The method of the present disclosure includes: preparing an extract through solvent extraction from a Ganoderma lucidum fruiting body; assaying the antithrombotic activity of the extract; and investigating the stability of the Ganoderma lucidum fruiting body ethanol extract.

The “Ganoderma lucidum fruiting body extract” included in the composition of the present disclosure can be obtained by drying the fruiting body of Ganoderma lucidum after harvesting the fruiting body of Ganoderma lucidum, cutting the fruiting body into fragments with a size of 2 to 3 cm, extracting the fragments with an organic solvent, filtering the resulting extraction solution with a filter with a mesh of 0.06 mm or less, and performing vacuum concentration on the filtrate.

Examples of the organic solvent that can be used in the present disclosure include water (cold water or hot water), hexene, methylene chloride, acetone, alcohol, anhydrous or hydrous lower alcohol having 1 to 4 carbon atoms (methanol, ethanol, alcohol, propanol, butanol, etc.), a mixture of the lower alcohol and water. Preferably, 95% ethanol extraction is preferred.

The ethanol extract may be sequentially or individually fractionated with organic solvents of hexene, ethyl acetate, and butanol to additionally obtain a hexene fraction, an ethyl acetate fraction, and a butanol fraction, and a water residue.

In the present disclosure, thrombin time, prothrombin time, and aPT time were measured from Ganoderma lucidum fruiting body extract at a concentration of 5 mg/ml. As a result, it was confirmed that ethanol extracts of Ganoderma lucidum fruiting body exhibited very strong thrombin inhibitory activity, prothrombin inhibitory activity, and coagulation factor inhibitory activity. The inhibitory activity of thrombogenesis of the extract was anticoagulant activity that was better than that of aspirin (1.5 mg/ml), which is used as an antithrombotic agent in clinical practice. In addition, it was confirmed that the extract could be practically used as a preventive or therapeutic/ameliorating agent for thrombosis because it had no hemolytic activity on human erythrocytes and had excellent thermal stability and acid stability so as to be taken in various forms.

The Ganoderma lucidum fruiting body extract of the present disclosure can be prepared in the form of powder through a conventional powdering process such as vacuum drying, freeze drying, or spray drying. The extract is not degraded by various degrading enzymes in plasma, and maintain its activity even under conditions such as a heat treatment temperature of 100° C. and pH 2 which is the pH of the human stomach.

The active ingredient of the present disclosure can be used for the prevention or treatment of various diseases related to thrombosis. For example, these diseases include: arterial thrombosis such as acute myocardial infarction, chest pain, dyspnoea, loss of consciousness, ischemic stroke, hemorrhagic stroke, headache, dyskinesia, paresthesia, personality changes, blurred vision, epileptic seizures, pulmonary thrombosis, deep vein thrombosis, lower limb edema, pain and acute peripheral arterial occlusion, and the like; and venous thrombosis such as deep vein thrombosis, portal vein thrombosis, acute renal vein occlusion, cerebral venous thrombosis, and central retinal vein occlusion.

The pharmaceutical composition containing the active ingredient of the present disclosure may be formulated by conventional methods and used in a variety of forms, including injectables such as sterile injectable solutions, and oral formulations such as powders, granules, tablets, capsules, suspensions, emulsions, syrups, and aerosols, for each purpose of use thereof.

The pharmaceutical composition may further include carriers, excipients, or diluents. Examples of the carrier, excipient, and diluent suitable for formulation include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil. In addition, a filler, an anti-agglomeration agent, a lubricant, a wetting agent, a flavoring agent, a preservative, and the like may be additionally contained in the pharmaceutical composition of the present disclosure.

As a preferred embodiment, solid formulations for oral administration include tablets, pills, powders, granules, capsules, and the like. These solid formulations are formulated by adding at least one excipient such as starch, calcium carbonate, sucrose, lactose, gelatin, and the like to the pharmaceutical composition. In addition, besides simple excipients, lubricants such as magnesium stearate, talc, or the like may be used.

As a referred embodiment, oral liquid preparations may include suspensions, solutions, emulsions, and syrups. In addition to water and liquid paraffin, which are commonly used simple diluents, various excipients such as wetting agents, sweetening agents, fragrances, preservatives, and the like may be included in oral liquid preparations.

As a preferred embodiment, preparations for parenteral administration include sterilized aqueous solutions, non-aqueous solutions, suspensions, emulsions, lyophilized formulations, suppositories, and the like. As the non-aqueous solvent and the suspending agent, propylene glycol, polyethylene glycol, vegetable oils such as olive oil, injectable esters such as ethyloleate, and the like may be used. Injectables may include conventional additives such as solubilizers, isotonic agents, suspending agents, emulsifiers, stabilizers, preservatives, and the like.

The active ingredient of the present disclosure is administered in a pharmaceutically effective amount. In the present disclosure, the pharmaceutically effective amount means an amount that is sufficient to treat a disease with a reasonable benefit/risk ratio applicable to medical treatment, and the effective dosage level depends on various factors including the type and severity of a disease, drug activity on the disease, patient's sensitivity to drug, administration time, administration route, excretion rate, treatment period, and co-used drugs and other factors well known in the medical field. The pharmaceutical composition according to the present disclosure may be administered as an individual therapeutic agent or administered in combination with other therapeutic agents. When administered in combination, the composition and other therapeutic agents may be administered sequentially or simultaneously. The composition may be administered as a single dose or multiple doses. In consideration of all of the above factors, it is important to administer a dosage that can obtain the maximum efficacy with a minimum amount without side effects, and the dosage can be easily determined by those skilled in the art.

As a preferred embodiment, the effective amount of the active ingredient in the pharmaceutical composition of the present disclosure may vary depending on the patient age, sex, and weight. Generally, the effective amount of the active ingredient may be 1 to 5,000 mg and preferably 100 to 3,000 mg per kg of body weight. The effective amount may be administered daily or every other day, or may be divided and administered 1 to 3 times a day. However, the effective amount does not in any way limit the scope of the present disclosure because it can be increased or decreased depending on the route of administration, the severity of disease, the patient sex, weight, age, and the like, etc.

The pharmaceutical composition of the present disclosure may be administered to a subject through various routes. All modes of administration can be envisaged: oral administration; rectal or intravenous administration; and intramuscular, subcutaneous, intrauterine intrathecal, or intracerebroventricular injection.

In the present disclosure, “administration” means providing a predetermined substance to a patient by any suitable method, and the route of administration of the pharmaceutical composition of the present disclosure may be oral or parenteral routes through which the composition can reach the target tissue. In addition, the composition of the present disclosure may be administered using any device capable of delivering active ingredients to target cells.

In the present invention, the term “subject” refers to, but is not particularly limited to, for example, human, monkey, cow, horse, sheep, pig, chicken, turkey, quail, cat, dog, mouse, rat, rabbit or guinea pig. The subject refers to preferably a mammal and more preferably a human.

In addition, the health functional food of the present disclosure may be used in a variety of ways, such as foods and beverages which are effective for preventing or improving thrombosis. Foods containing the active ingredient of the present disclosure include, for example, various foods, beverages, gum, tea, vitamin complexes, health supplements, etc. The foods can be used in the form of powders, granules, tablets, capsules, or beverages.

The active ingredient of the present disclosure may be overall added in an amount of 0.01% to 15% by weight with respect to the total food weight, and a health drink composition may contain the active ingredient of the present disclosure in an amount of 0.02 g to 10 g and preferably 0.3 g to 1 g, based on 100 ml thereof.

The health functional food of the present disclosure may contain food auxiliary additives such as natural carbohydrates and various flavoring agents as additional ingredients in addition to containing the compounds as essential ingredients in the predetermined ratios.

Examples of the natural carbohydrate include monosaccharides such as glucose and fructose, disaccharides such as maltose and sucrose, polysaccharides such as dextrin and cyclodextrin, and sugar alcohols such as xylitol, sorbitol, and erythritol.

As the flavoring agent, natural flavoring agents such as stevia such as thaumatin, rebaudioside A or glycyrrhizin, and synthetic flavoring agents such as saccharin and aspartame may be used. The proportion of the natural carbohydrate is generally about 1 to 20 g and preferably about 5 to 12 g, per 100 ml of the health functional food of the present disclosure. Aside from the ingredients described above, the health functional food of the present disclosure may further include various nutrients, vitamins, minerals, flavors such as synthetic flavors and natural flavors, colorants and color enhancers, pectic acid and salts thereof, alginic acid and salts thereof, organic acids, protective colloidal thickeners, pH adjusters, stabilizers, preservatives, glycerin, alcohols, carbonation agents used in carbonated beverages, and the like. In addition, the health functional food of the present disclosure may contain fruit flesh for production of natural fruit juice, fruit juice beverages, and vegetable beverages. These ingredients may be used solely or in combination. The ratio of these additives is generally selected in the range of 0.01 to about 20 parts by weight per 100 parts by weight of the active ingredient of the present disclosure.

The present disclosure will be described in more detail with reference to examples below. The examples described below are only preferred embodiments of the present disclosure, and the scope of the present disclosure is not limited by the examples.

### EXAMPLE

### Example 1: Ganoderma lucidum Fruiting Body Acquisition and Chrominance Analysis

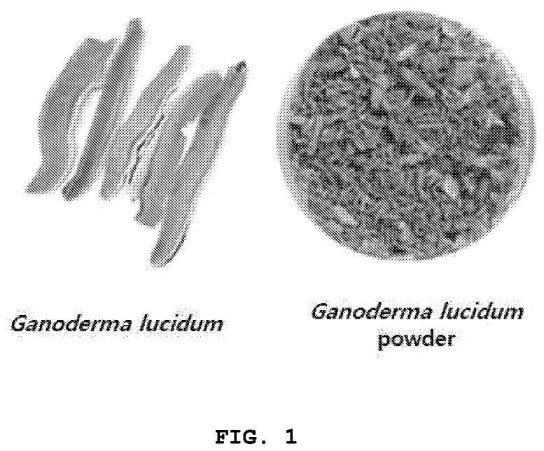

The fruiting body of Ganoderma lucidum was provided by Gold Palm Bio Co., Ltd. in Mungyeong, Gyeongsangbuk-do, Korea. The shape of Ganoderma lucidum and the chrominance analysis results of Ganoderma lucidum after grinding are shown in FIG. 1 and Table 1.

TABLE 1
Color analysis of Ganoderma lucidum fruiting body.

| | | | Color difference | | | |
|---|---|---|---|---|---|---|
| | | | | | | Overall |
| | | | | | | color |
| | | | Lightness | Redness | Yellowness | difference |
| Extract | pH | brix | (L) | (a) | (b) | (Δ E) |
| Ganoderma | 4.8 | 3.6 | 33.66 ± | 6.72 ± | 13.28 ± | 60.19 ± |
| lucidum | | | 0.19 | 0.09 | 0.08 | 0.16 |

L: degree of lightness(white +100~0 black),
a: degree of redness(red +100~−80 green),
b: degree of yellowness(yellow +70~−80 black),
Δ E: overall color difference.

The pH of the Ganoderma lucidum was 4.8, and the degree of Brix indicating the concentration of total soluble solids was 3.6. In addition, the lightness (L) was 33.66, the redness (a) was 6.72, the yellowness (b) was 13.28, and the overall color difference was 60.19.

### Example 2: Preparation of Ethanol Extract of Ganoderma lucidum and Component Analysis of Extract

An ethanol extract was prepared from the powder of Ganoderma lucidum fruiting body prepared in Example 1. 10-fold ethanol (95%, Ducksan, Korea) was added to the sample, and room temperature extraction was performed two times on the same sample. The extracted of the respective extractions were collected and filtered. The filtrate was vacuum concentrated to prepare powder. The extraction yields and useful ingredient analysis results of Ganoderma lucidum mushroom fruiting bodies are shown in Table 2.

TABLE 2
Yield and useful ingredients of extract
of Ganoderma lucidum fruiting body.

| | Extrac- | | | | |
|---|---|---|---|---|---|
| | tion ef- | Content(mg/g) | | | |
| | ficiency | Total | Total | Total | Reducing |
| Extract | (%) | polyphenol | flavonoid | sugar | sugar |
| Ganoderma | 1.2 | 59.1 ± 2.7 | 6.1 ± 0.7 | 49.7 ± 0.9 | 23.0 ± 0.9 |
| lucidum | | | | | |

As shown in Table 2, the extraction efficiency of the Ganoderma lucidum fruiting body was 1.2%, which indicated a high woody content. The contents of total polyphenols, total flavonoids, total sugars, and reducing sugars were measured for the useful ingredient analysis of each of the Ganoderma lucidum fruiting body extracts. In this case, for the determination of the total polyphenol content, 50 μl of Folin-ciocalteau and 100 μl of Na₂CO₃ saturated solution were added to 400 μl of an extract sample solution, and the mixture was rested for 1 hour, followed by the measurement of absorbance at 725 nm. As a standard reagent, tannic acid was used. For the determination of the total flavonoid content, each sample was stirred and extracted with ethanol for 18 hours, 4 ml of 90% diethylene glycol was added to 400 μl of a filtered extract sample solution, 40 μl of 1 N NaOH was added thereto, followed by reaction at 37° C. for 1 hour, and the absorbance was measured at 420 nm. Rutin was used as a standard reagent.

Total sugar was quantified using the phenol-sulfuric acid method, and sucrose was used as a standard reagent. For the quantification of reducing sugar, the DNS method was used, and glucose was used as a standard reagent.

Analysis of the total polyphenol content and the total flavonoid content revealed a high polyphenol content of 59.1 mg/g and a flavonoid content of 6.1 mg/g. However, as a result of analyzing the total sugar content and the reducing sugar content, the total sugar content was 49.7 mg/g and the reducing sugar content was 22.0 mg/g, so it was expected that the Ganoderma lucidum fruiting body extract would exhibit various useful physiological activities.

Given the extraction efficiency, the fruiting body of Ganoderma lucidum was expected to contain 71 mg of polyphenols per 100 g of the fruiting body.

### Example 3: Evaluation of Blood Coagulation Inhibitory Activity of Ganoderma lucidum Fruiting Body Extract

The blood coagulation inhibitory activity of the Ganoderma lucidum fruiting body extract of Example 2 was evaluated and the results are shown in Table 3. The blood coagulation inhibitory activity of Ganoderma lucidum fruiting body samples was evaluated according to the previously reported methods (Sohn et al., 2004. Kor. J. Pharmacogn 35. 52-61; Kwon et al., 2004. J Life Science, 14. 509-513; Ryu et al. 2010. J. Life Science, 20. 922-928). For this, thrombin time, prothrombin time and aPT time were measured. For plasma, commercially available control plasma (MD Pacific Technology Co., Ltd, Huayuan Industrial Area, China) was used, and thrombin time, prothrombin time, and aPT time measurements were performed in a manner described below.

Thrombin Time

At 37° C., 50 μl of 0.5 U thrombin (Sigma Co., USA), 50 μl of 20 mM CaCl₂, and 10 μl of sample extracts of various concentrations were mixed in the tube of Amelung coagulometer KC-1 A (Japan), and reacted for 2 minutes, followed by addition of 100 μl of plasma. After the addition of plasma, the time until plasma coagulation was measured. As a control, aspirin (Sigma Co., USA) was used, and as a solvent control, DMSO was used instead of the sample. In the case of DMSO, the coagulation time was 32.1 seconds.

The thrombin inhibitory activity was expressed with the average of the results of three or more repeated experiments, and the thrombin inhibitory activity was expressed as the value obtained by dividing the coagulation time upon addition of the sample by the coagulation time upon addition of the solvent control.

Prothrombin Time

70 μl of standard plasma (MD Pacific Co., China) and 10 μl of sample solutions of various concentrations were added to the tube of Amelung coagulometer KC-1A (Japan) and warmed at 37° C. for 3 minutes, followed by addition of 130 μl of PT reagent. After the addition of the PT reagent, the time until coagulation was measured. The time was expressed with the average value of the results of three repeated experiments. As a control, aspirin (Sigma Co., USA) was used, and as a solvent control, DMSO was used instead of the sample. In the case of DMSO, the coagulation time was 18.1 seconds. The prothrombin inhibitory activity was expressed as the value obtained by dividing the coagulation time upon addition of the sample by the coagulation time of the solvent control.

aPTT (Activated Partial Thromboplastin Time)

100 μl of plasma and 10 μl of sample extracts of various concentrations were added to the tube of Amelung coagulometer KC-1A (Japan) and warmed at 37° C. for 3 minutes. 50 μl of aPTT reagent (Sigma, ALEXIN™) was added thereto and incubated at 37° C. for 3 minutes. 50 μl of CaCl₂ (35 mM) was added, and the time until plasma coagulation was measured. As a solvent control, DMSO was used instead of the sample, and in this case, the coagulation time was 55.1 seconds. The aPTT was expressed with the average of the results of three or more repeated experiments, and the blood coagulation factor inhibitory activity was expressed as the value obtained by dividing the aPTT upon addition of the same by the aPTT time upon addition of the solvent control.

TABLE 3
Blood coagulation inhibitory activity of
Ganoderma lucidum fruiting body extract.

| | Concen- | Blood coagulation inhibitory activity* | | |
|---|---|---|---|---|
| | tration | Thrombin | Prothrombin | |
| Control/Extract | (mg/ml) | time | time | aPT time |
| Solvent control | — | 1.00 ± 0.07 | 1.00 ± 0.01 | 1.00 ± 0.00 |
| (DMSO) | | | | |
| Aspirin | 1.5 | 1.45 ± 0.02 | 1.40 ± 0.00 | 1.57 ± 0.11 |
| Ganoderma | 5 | 1.71 ± 0.04 | 1.74 ± 0.04 | 3.05 ± 0.12 |
| lucidum | 2.5 | 1.48 ± 0.01 | 1.45 ± 0.11 | 1.91 ± 0.15 |
| fruiting body | | | | |

Blood coagulation inhibitory activity: Blood coagulation time of sample added group/Blood coagulation time of DMSO added group

As shown in Table 3, as a result of measuring the thrombin time, prothrombin time, and aPT time using the prepared ethanol extract of Ganoderma lucidum fruiting bodies at a concentration of 5 mg/ml, the thrombin time, the prothrombin time, and the aPT time were prolonged 1.71, 1.74, and 3.05 times, respectively. This means that considerably strong antithrombotic activity was exhibited. Even when the ethanol extract of Ganoderma lucidum fruiting bodies was evaluated at a concentration of 2.5 mg/ml, the thrombin time, the prothrombin time, and the aPT time were extended by 1.48, 1.45, and 1.91 times, respectively. This means that the antithrombotic activity of the extract is stronger than that of aspirin (1.5 mg/ml) used clinically as an antithrombotic agent.

Given that Ganoderma lucidum fruiting bodies are currently consumed mainly as teas and beverages, the Ganoderma lucidum fruiting body extract is expected to be able to effectively inhibit thrombogenesis (blood clot formation) through the inhibition of various blood coagulation factors, thrombin, and prothrombin, and is expected substitute for aspirin exhibiting severe side effects such as gastrointestinal disorders.

### Example 4: Hemolytic Activity of Ganoderma lucidum Fruiting Body Extract on Human Erythrocyte

In order to evaluate the possibility of acute toxicity of the Ganoderma lucidum fruiting body extract prepared in Example 2, the hemolytic activity on human erythrocytes was evaluated. The hemolytic activity was evaluated according to the previous report (Son Ho-yong, 2014. Korean J. Microbiol. Biotechnol. 42: 285-292). Briefly, 100 μl of human red blood cells washed three times with PBS were placed in a 96-well microplate, 100 μl of sample solution of various concentrations was added thereto, the reaction was performed at 37° C. for 30 minutes, and the reaction solution was centrifuged at 1,500 rpm for 10 minutes, 100 μl of the supernatant was transferred to a new microtiter plate, and the degree of hemoglobin leakage due to hemolysis was measured at 414 nm. DMSO (2%) was used as a solvent control for the sample, and Triton X-100 (1 mg/ml) was used as a control for red blood cell hemolysis. Hemolytic activity was calculated using the following formula:

(%)Hemolysis=[(Abs.S−Abs.C)/(Abs.T−Abs.C)]×100
Abs.S: Absorbance of sample added group
Abs.C: Absorbance of DMSO added group
Abs.T: Absorbance of Triton X-100 added group

TABLE 4
Hemolytic activity of Ganoderma lucidum
fruiting body extract on human erythrocyte.

| | Concentration | Human erythrocyte |
|---|---|---|
| Sample/Control | (mg/ml) | coagulation activity(%) |
| Distilled water | — | 0.2 ± 0.2 |
| Solvent(DMSO) | | |
| Triton X-100 | — | 0.4 ± 0.6 |
| Amphotericin B | 1.0 | 100.0 ± 0.1 |
| | 0.1 | 99.6 ± 1.0 |
| | 0.05 | 99.6 ± 1.2 |
| | 0.025 | 98.3 ± 0.8 |
| | 0.0125 | 96.9 ± 0.0 |
| | 0.0063 | 89.1 ± 1.5 |
| | 0.0032 | 61.0 ± 0.1 |
| | 0 | 0.0 ± 0.1 |
| Ganoderma lucidum | 1.0 | 51.6 ± 2.4 |
| fruiting body extract | 0.5 | 34.1 ± 0.3 |
| | 0.25 | 12.6 ± 0.2 |
| | 0.13 | −1.5 ± 0.8 |

As shown in Table 4, it was confirmed that DMSO and water used as controls did not exhibit hemolytic activity, and Triton X-100 hemolyzed 1000 of red blood cells at a concentration of 1 mg/ml. In addition, in the case of amphotericin B, which is used as an anticancer and antifungal agent, it was confirmed that more than 50% of red blood cells were hemolyzed at a concentration of 0.0032 mg/ml. On the other hand, the fruiting body extract of Ganoderma lucidum showed 51.6% of erythrocyte hemolysis at a concentration of 1 mg/ml. It is assumed that the hemolytic activity of the Ganoderma lucidum fruiting body extract is attributed to saponin, etc.. Given that the Ganoderma lucidum fruiting body extract has been used for food (extracted tea, beverage containing several extracts), the extract has no serious acute toxicity. In particular, compared to amphotericin B, which has side effects, it was determined that acute toxicity to humans would not appear because the extract of the fruiting body of Ganoderma lucidum required about 300 times higher concentration to show the same red blood cell hemolytic activity.

### Example 5: Evaluation of Plasma Stability, Acid Stability, and Thermal Stability of Ganoderma lucidum Fruiting Body Extract

The Ganoderma lucidum fruiting body extract prepared in Example 2 was prepared and consumed as ordinary teas, complex beverages, and alcoholic beverages, so it was determined that there would be no particular problem in safety. Therefore, plasma stability, thermal stability, and acid stability of the fruiting body extract of Ganoderma lucidum for blood coagulation inhibitory activity were confirmed. The extract did not show a decrease in blood coagulation inhibitory activity even when heat-treated at 100° C. for 1 hour, treated with a pH 2 acid (0.01 M HCl) for 1 hour, and treated with plasma for 1 hour. Therefore, it was confirmed that the Ganoderma lucidum fruiting body extract contained an antithrombotic substance having acid resistance and heat resistance, and thus, it was confirmed that the practical applicability thereof was high.

What is claimed is:

1. A pharmaceutical composition comprising a Ganoderma lucidum fruiting body extract as an active ingredient for preventing or treating thrombosis.

2. The pharmaceutical composition of claim 1, wherein the Ganoderma lucidum fruiting body extract is a Ganoderma lucidum fruiting body ethanol extract.

3. A blood coagulation inhibitor comprising the active ingredient of claim 1.

4. A health functional food comprising the active ingredient of claim 1, for preventing or ameliorating thrombosis.

5. A blood coagulation inhibitor comprising the active ingredient of claim 2.

6. A health functional food comprising the active ingredient of claim 2, for preventing or ameliorating thrombosis.
